(19) United States
(12) Patent Application Publication
(10) Pub. No.: US 2026/0097215 A1
(43) Pub. Date: Apr. 9, 2026

(54) SYSTEMS FOR DETERMINING LEAD MARKER ORIENTATION

(71) Applicant: Boston Scientific Neuromodulation Corporation, Valencia, CA (US)

(72) Inventors: Lisa Denise Moore, Glendale, CA (US); G. Karl Steinke, Valencia, CA (US); Mahsa Malekmohammadi, Sherman Oaks, CA (US)

(21) Appl. No.: 18/905,931

(22) Filed: Oct. 3, 2024

Related U.S. Application Data
(60) Provisional application No. 63/594,895, filed on Oct. 31, 2023.

(51) Int. Cl. A61N 1/372; A61N 1/05; A61N 1/36

(52) U.S. Cl. CPC A61N 1/372; A61N 1/0534; A61N 1/36135

(57) ABSTRACT

A system may include a lead including segmented electrodes positioned peripherally around the lead, and an orientation system configured to determine an orientation of the lead. The orientation system may include sensing circuitry configured to use the segmented electrodes positioned peripherally around the lead to sense signals from a signal generator, wherein the signal generator has a known location distinct from a location of the at least one neural target in the patient, and processing circuitry configured to determine at least one feature a strength of a sensed signal received at individual ones of the segmented electrodes positioned peripherally around the lead and to use the at least one feature at the individual ones of the segmented electrodes to determine lead orientation.

### CLAIM OF PRIORITY

This application claims the benefit of U.S. Provisional Application No. 63/594,895, filed on October 31, 2023, which is hereby incorporated by reference in its entirety.

### TECHNICAL FIELD

This document relates generally to medical systems, and more particularly, but not by way of limitation, to systems, devices, and methods for determining an orientation for a marker of a neuromodulation lead.

### BACKGROUND

Medical devices may include devices configured to deliver a therapy to a patient and/or systems configured to monitor a patient condition via user input and/or sensor(s). For example, these devices may include wearable devices and implantable devices. Some implantable devices may use one or more leads to sense electrical signals or to treat various biological disorders, such spinal cord stimulators (SCS) to treat chronic pain, cortical and Deep Brain Stimulators (DBS) to treat motor and psychological disorders, Peripheral Nerve Stimulation (PNS) including Vagus Nerve Stimulation (VNS), Functional Electrical Stimulation (FES), and other neural stimulators to treat urinary incontinence, sleep apnea, shoulder subluxation, and the like.

Lead(s) used to deliver stimulation may be directional leads, including radially directional leads, to provide a modulation field shape on one side of the lead to stimulate a neural target on that side of the lead without adversely stimulating other regions, which may also be referred to as avoidance regions, near the lead. Thus, knowing lead orientation, including lead rotational orientation, is useful for providing directional control of stimulation. Similarly, knowing lead orientation is useful in providing direction of sensing of electrical signals. Generally, a physician understands that certain targets and avoidance regions may be located more laterally or medially, or otherwise with some orientation with respect to the intended and achieved lead placement and orientation.

Physicians may rely on the lead marker orientation to help inform them during implant and patient programming. This may be particularly important for DBS directional leads. The lead marker may be detected using intraoperative fluoroscopy, intraoperative computer tomography (CT) or postoperative imaging, or other peri- or post-operative methods. Programming software may support directional programming by providing relative or absolute anatomical direction in the patient anatomical space. The anatomical directions presented to the user may be determined using logic based on the marker orientation, which is presently often determined based on the analysis of post-implant neuroimaging data and may be further manually adjusted by the clinician. The data may be used to target anatomy-based programming methods, but the algorithm implemented in the programming software may misidentify the orientation of the marker (e.g., by 180 degrees) as it may not be able to distinguish between a marker on a near surface of the lead or on a far surface of the lead, or otherwise the orientation in the software may be incorrect at some point in the workflow or point in time. Moreover, there is an inherent error margin associated with the analysis of immediate postoperative neuroimaging data due to pneumocephalus and image co-registration. These disparities can lead to confusion for the physician and introduce errors in anatomy-based programming algorithms, physiology-based programming algorithms, response-guided programming algorithms, or other programming aides.

The performance of any anatomy-based methods or algorithms used in an automated programming may be significantly impacted by inaccuracy in detecting the marker orientation. Additionally, inaccuracy in the marker orientation may cause manual programming responses to be misaligned to predicted outcomes as shown on the imported anatomy. This may lead to frustration from the users and a distrust in programming solutions. What is needed is an improved technique for determining the orientation of an implanted lead.

### SUMMARY

Various embodiments sense signals from a signal generator in a known location to aid in determining lead orientation. The signal generator may be a known or presumed signal generator. The signal generator may be internal or external to the patient. Internal signal generators may include machines or innate portions of a body that generate the signals, including various endogenous or natural signals or signal generators. Various embodiments provided herein use sensed physiological signals from a physiological or artificial signal generator to aid in lead orientation determination. The signal generator source may be an evoked signal generator. For example, stimulation of a location in the patient may cause an evoked signal from the signal generator. The location of the signal generator may be different from the stimulated location. The evoked signal may be stimulated or sensed at various locations in the body. For example, stimulating an area of the body may evoke a neural response, may generate sensory evoked potentials, or may generate motor evoked potentials. Some embodiments may incorporate stimulation-induced side effect (e.g., facial muscle pulling below a visual perception level, as sensed using electromyography EMG).

An example (e.g., “Example 1”) of a system may include a lead including segmented electrodes positioned peripherally around the lead, and an orientation system configured to determine an orientation of the lead implanted in a patient. The orientation system may include sensing circuitry configured to use the segmented electrodes positioned peripherally around the lead to sense signals from a signal generator, wherein the signal generator has a known location distinct from a location of the at least one neural target in the patient, and processing circuitry configured to determine at least one feature a strength of a sensed signal received at individual ones of the segmented electrodes positioned peripherally around the lead and to use the at least one feature at the individual ones of the segmented electrodes to determine lead orientation.

In Example 2, the subject matter of Example 1 may optionally be configured such that the lead includes a lead marker at a known position on the lead and useful for determining an orientation of the lead, the orientation system includes a waveform generator configured to deliver an electrical waveform to stimulate at least one neural target according to neural stimulation parameters, and the signal generator has a known location distinct from a location of the at least one neural target in the patient. The system may a medical imaging system configured to detect the lead and to detect the lead marker on the lead (or may be configured to receive lead marker imaging information from the medical imaging system). The processing circuitry may be configured to use both the at least one feature of the sensed signal and the detected lead marker to determine the lead orientation, and to determine the stimulation parameters for stimulating the at least one neural target using the determined lead orientation.

In Example 3, the subject matter of any one or more of Examples 1-2 may optionally be configured such that the orientation system includes a neural stimulator system, and the neural stimulator system includes: an external system configured for use by a clinician during placement of the lead or other implanted component; or an implantable device and the sensing circuitry is within the implantable device.

In Example 4, the subject matter of any one or more of Examples 1-3 may optionally be configured such that the signal generator is innate in the patient.

In Example 5, the subject matter of Example 4 may optionally be configured such that the signal generator is an evoked response generator configured to generate evoked response signal in response to stimulation.

In Example 6, the subject matter of Example 5 may optionally be configured such that the generated evoked responses include evoked motor potentials, evoked neural responses, or sensory-evoked signals.

In Example 7, the subject matter of any one or more of Examples 1-3 may optionally be configured such that the signal generator includes a machine or device (e.g., artificial or non-physiological) positioned externally or internally in the patient.

In Example 8, the subject matter of any one or more of Examples 1-7 may optionally be configured such that the lead includes a deep brain stimulation (DBS) lead configured for implantation in a brain and the signal generator is in the brain.

In Example 9, the subject matter of Example 8 may optionally be configured such that the generated evoked responses include evoked resonant neural activity (ERNA) in the brain.

In Example 10, the subject matter of any one or more of Examples 2-9 may optionally be configured to further include delivering neurostimulation to the at least neural target using the determined stimulation parameters for the determined lead orientation.

In Example 11, the subject matter of any one or more of Examples 1-10 may optionally be configured such that the lead marker is detected using medical imaging before the evoked responses are sensed, the detected lead marker on the lead is used to provide a suggested orientation of the lead, and the sensed signals from the known position of the evoked response generator are used to confirm the suggested orientation of the lead or refine or modify the suggested orientation of the lead.

In Example 12, the subject matter of any one or more of Examples 1-10 may optionally be configured such that the evoked responses are sensed before the lead marker is detected using medical imaging, and the processing circuitry is configured to automatically provide a recommendation for rotating the lead during a lead implantation procedure.

In Example 13, the subject matter of any one or more of Examples 1-12 may optionally be configured such that the lead is a first lead, and the orientation system is configured to sense the signals from the signal generator using segmented electrodes positioned peripherally around a second lead and determine the lead orientation for the first lead is determined using the signals sensed using the second lead.

In Example 14, the subject matter of any one or more of Examples 1-13 may optionally be configured such that the first lead is a first DBS lead and the second lead is a second DBS lead, and the first DBS lead and the second DBS lead are contralateral leads.

In Example 15, the subject matter of any one or more of Examples 1-13 may optionally be configured such that the first lead is a first DBS lead and the second lead is a second DBS lead, and the first DBS lead and the second DBS lead are in a same hemisphere of the brain.

Example 16 includes subject matter (such as a method, means for performing acts, machine readable medium including instructions that when performed by a machine cause the machine to perform acts, or an apparatus to perform). The subject matter may include determining an orientation of a lead for stimulating at least one neural target in a patient and determining stimulation parameters for stimulating the at least one neural target using the determined lead orientation.

In Example 17, the subject matter of Example 16 may optionally be configured such that the signal generator is a known evoked response generator in the patient and the signals sensed from the signal generator include evoked response signals, and the method further includes causing the known evoked response generator to generate the evoked response signals.

In Example 18, the subject matter of Example 17 may optionally be configured such that the lead is used to deliver stimulation to cause the known evoked response generator to generate the evoked response signals.

In Example 19, the subject matter of Example 18 may optionally be configured such that the generated evoked responses include evoked motor potentials.

In Example 20, the subject matter of Example 18 may optionally be configured such that the generated evoked responses include evoked neural responses.

In Example 21, the subject matter of Example 18 may optionally be configured such that the generated evoked responses include sensory-evoked signals.

In Example 22, the subject matter of any one or more of Examples 16-21 may optionally be configured such that the lead includes a deep brain stimulation (DBS) lead configured for implantation in a brain.

In Example 23, the subject matter of Example 22 may optionally be configured such that the location of the known evoked response generator is in the brain.

In Example 24, the subject matter of Example 16 may optionally be configured such that the lead includes a deep brain stimulation (DBS) lead configured for implantation in a brain and the generated evoked responses include evoked resonant neural activity (ERNA) in the brain.

In Example 25, the subject matter of any one or more of Examples 16-24 may optionally be configured to further include delivering neurostimulation to the at least neural target using the determined stimulation parameters for the determined lead orientation.

In Example 26, the subject matter of any one or more of Examples 16-25 may optionally be configured such that the lead marker is detected using medical imaging before the evoked responses are sensed.

In Example 27, the subject matter of Example 26 may optionally be configured such that the detected lead marker on the lead is used to provide a suggested orientation of the lead, and the sensed signals from the known position of the evoked response generator are used to confirm the suggested orientation of the lead or modify the suggested orientation of the lead.

In Example 28, the subject matter of Example 16-27 may optionally be configured such that the evoked responses are sensed before the lead marker is detected using medical imaging.

In Example 29, the subject matter of Example 28 may optionally be configured to further include automatically providing a recommendation for rotating the lead during a lead implantation procedure.

In Example 30, the subject matter of Example 16-27 may optionally be configured to further include using the medical imaging to implant the lead into position to stimulate the at least one neural target.

In Example 31, the subject matter of Example 30 may optionally be configured to further include orientating the lead into a desired orientation using both the strength of the sensed signal and the detected lead marker while the lead is being implanted.

In Example 32, the subject matter of Example 16-27 may optionally be configured such that the lead is a first lead and the subject matter further includes sensing the evoked responses from the evoked response generator using segmented electrodes positioned peripherally around a second lead. The lead orientation for the first lead may be determined using the evoked response sensed using the second lead.

In Example 33, the subject matter of Example 16-32 may optionally be configured such that the first lead is a first DBS lead and the second lead is a second DBS lead, and the first DBS lead and the second DBS lead are contralateral leads.

In Example 34, the subject matter of Example 16-32 may optionally be configured such that the first lead is a first DBS lead and the second lead is a second DBS lead, and the first DBS lead and the second DBS lead are in a same hemisphere of the brain.

Example 35 includes subject matter that includes non-transitory machine-readable medium including instructions, which when executed by a machine, cause the machine to perform a method. The method performed by the machine may include determining an orientation of a lead for stimulating at least one neural target in a patient and determining stimulation parameters for stimulating the at least one neural target using the determined lead orientation. The lead may include segmented electrodes positioned peripherally around the lead and a lead marker at a known position on the lead and useful for determining the orientation of the lead. The orientation of the lead may be determined by receiving lead marker information indicative of a detected lead marker on the lead that was detected using medical imaging, sensing signals from a signal generator using the segmented electrodes positioned peripherally around the lead where the known signal generator has a known location distinct from a location of the at least one neural target in the patient, determining a strength of a sensed signal received at individual ones of the segmented electrodes positioned peripherally around the lead, and using both the strength of the sensed signal and the detected lead marker to determine the lead orientation. In further examples, the subject matter of Example 31 may be configured such that the method performed by the machine may include any of the subject matter recited in Examples 16-34.

Some embodiments may use an orientation system additionally to or instead of a neural stimulation system. For example, the orientation system may be operating room equipment designed to determine orientation but may not necessarily be capable of delivering neurostimulation or at least therapeutically good neurostimulation. Some embodiments may also be configured to sense signals from the signal generator and determine one or more features of the signal (e.g., amplitude, temporal features or spectral features) and use the feature(s) to determine the orientation of the lead. In some embodiments, the orientation is determined only using the sensed features. An example of a system may include a lead and an orientation system for determining the orientation of the lead. The orientation system may include sensing circuitry and processing circuitry. The sensing circuitry may be configured to use the segmented electrodes positioned peripherally around the lead to sense signals from a signal generator. The processing circuitry may be configured to determine a signal feature(s) of a sensed signal received at individual ones of the segmented electrodes positioned peripherally around the lead. The processing circuitry may be configured to use the signal feature(s) of the sensed signal to determine the lead orientation and to determine the stimulation parameters for stimulating the at least one neural target using the determined lead orientation. Some embodiments may further include a lead marker on the lead, and medical imaging may be used to detect the lead and to detect the lead marker on the lead. The image-based detection of the lead marker may be used with the sensed features to determine a lead orientation. Features for various examples provided above or in this disclosure may be combined with this orientation system example. By way of example and not limitation, the orientation system may include a stimulator configured to evoke a response from the signal generator (e.g., ENRA).

An example (e.g., “Example 36”) of a system may include a lead for stimulating at least one neural target in a patient, a neural stimulator system and a medical imaging system. The lead may include segmented electrodes positioned peripherally around the lead and a lead marker at a known position on the lead and useful for determining the orientation of the lead. The neural stimulator system may include a waveform generator, sensing circuitry, and processing circuitry. The waveform generator may be configured to deliver an electrical waveform to stimulate the at least one neural target according to neural stimulation parameters. The sensing circuitry may be configured to use the segmented electrodes positioned peripherally around the lead to sense signals from a signal generator. The signal generator has a known location distinct from a location of the at least one neural target in the patient. The processing circuitry may be configured to determine a strength of a sensed signal received at individual ones of the segmented electrodes positioned peripherally around the lead. The medical imaging system may be configured to detect the lead and to detect the lead marker on the lead. The processing circuitry may be configured to use both the strength of the sensed signal and the detected lead marker to determine the lead orientation and to determine the stimulation parameters for stimulating the at least one neural target using the determined lead orientation.

In Example 37, the subject matter of Example 36 may optionally be configured such that the neural stimulator system is an external system configured for use by a clinician during placement of the lead or other implanted component, such as a later-placed waveform generator implanted during a revision or replacement procedure.

In Example 38, the subject matter of Example 36 may optionally be configured such that the neural stimulator system includes an implantable device and the waveform generator is within the implantable device.

In Example 39, the subject matter of any one or more of Examples 36-38 may optionally be configured such that the signal generator is innate in the patient.

In further examples, the subject matter of any one of Examples 36-39 may be configured to further include subject matter recited in Examples 4-15.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example and not limitation, a more generalized example of a medical system that includes a medical device and a processing system.

FIG. 6 illustrates, by way of example, an example of an electrical therapy-delivery system.

FIG. 7 illustrates, by way of example and not limitation, a monitoring system and/or the electrical therapy-delivery system of FIG. 6, implemented using an IMD.

FIG. 8 illustrates, by way of example and not limitation, an embodiment of a system for determining an orientation of a neurostimulation lead for use to stimulate a neural target based on sensed signal from a signal generator.

FIG. 9 illustrates, by way of example and not limitation, an embodiment of a method for determining an orientation of a neurostimulation lead for use to stimulate a neural target based on sensed signal from a signal generator.

FIG. 10 illustrates, by way of example and not limitation, an uncertainty in the marker position based solely on medical imaging, and the use of a signal generator such as an ERNA generator to resolve the uncertainty.

FIG. 11 illustrates, by way of example and not limitation, the use of a signal generator such as an ERNA generator to estimate a marker orientation and provide a recommendation for rotating the lead.

FIG. 12 illustrates, by way of example and not limitation, an estimated lead orientation based on medical imaging and a different estimated lead orientation based on sensed electrophysiological signals from a signal generator at a known location.

FIG. 13 illustrates, by way of example and not limitation, an embodiment of a method for revising an image-determined orientation estimate using a sensed signal sensed by the lead.

FIG. 14 illustrates, by way of example and not limitation, an embodiment of a system for determining an orientation of a neurostimulation lead for use to stimulate a neural target based on sensed signal from a signal generator as sensed by one or both of a first lead or a second lead.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized, and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

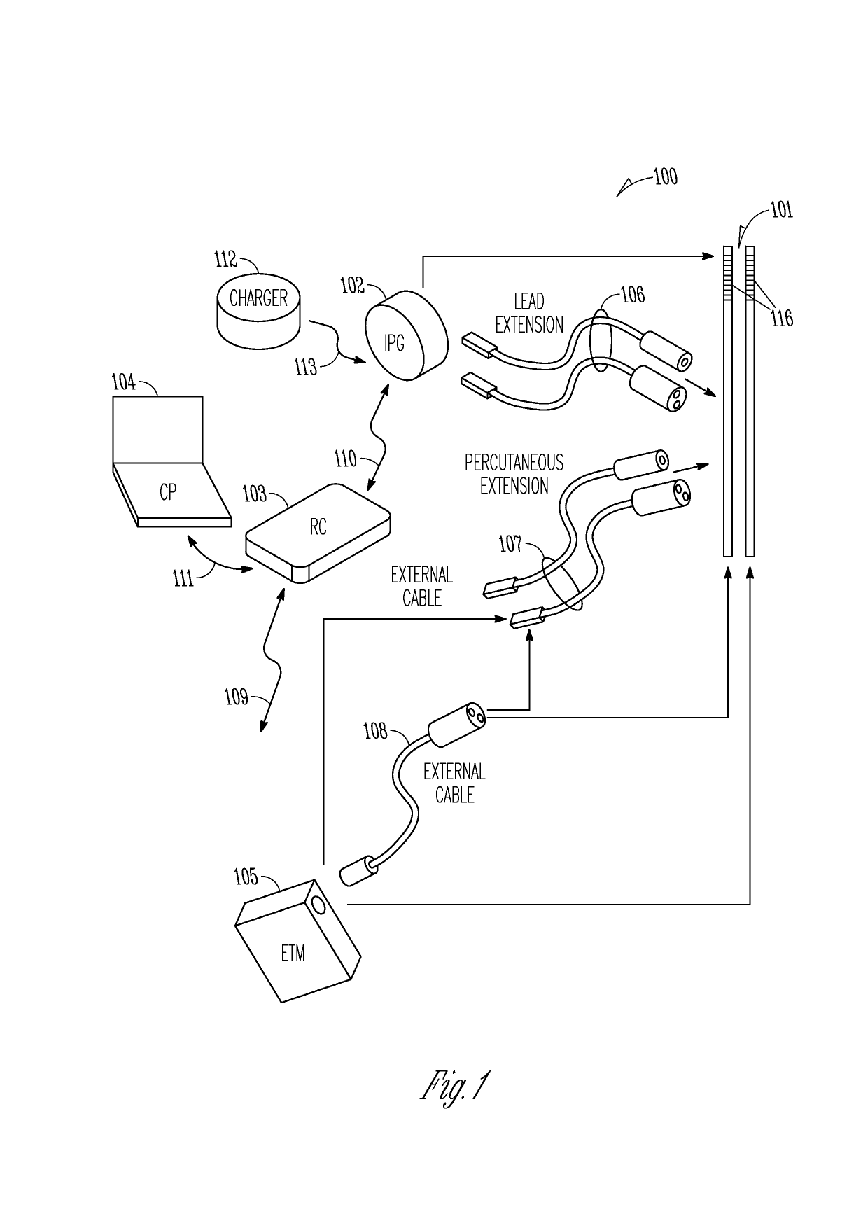

FIG. 1 illustrates, by way of example and not limitation, an electrical stimulation system 100, such as may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Other types of leads may be used. The IPG 102 includes pulse generation circuitry that delivers electrical modulation energy in the form of a pulsed electrical waveform (i.e.,a temporal series of electrical pulses) to the electrodes in accordance with a set of modulation parameters. The leads may be directional leads with a lead marker for use to help determine an orientation for an implanted lead. Directional leads may be used to target neural stimulation for DBS, SCS, PNS or other electrical stimulation.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. The ETM 105 may be used for situations where a brief period of therapy is suitable to achieve the desired effects. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned ON or OFF and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, the IPG 102 may function as programmed without the RC 103 or CP 104 being present. It is noted that some IPGs do not require charging, as some are manufactured with primary batteries with sufficient capacity to provide therapy over a clinically useful duration without recharging.

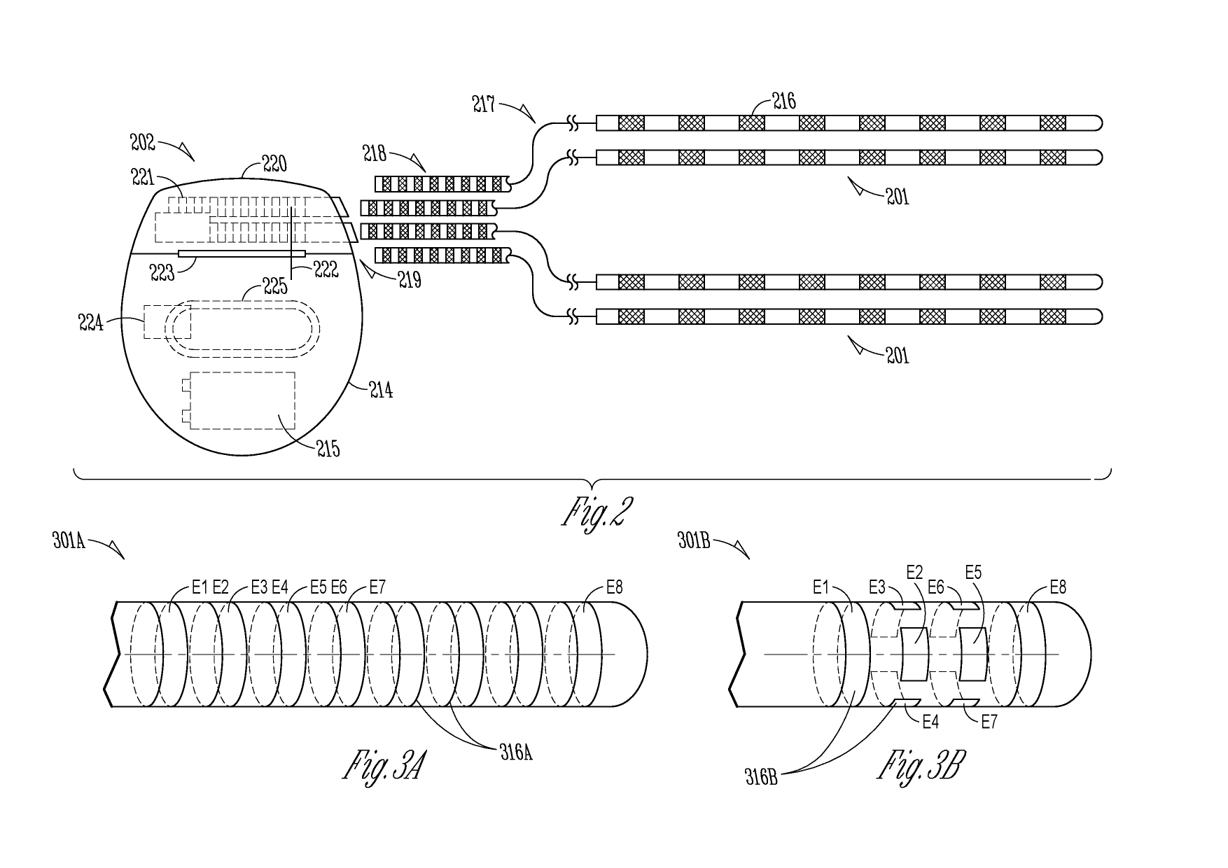

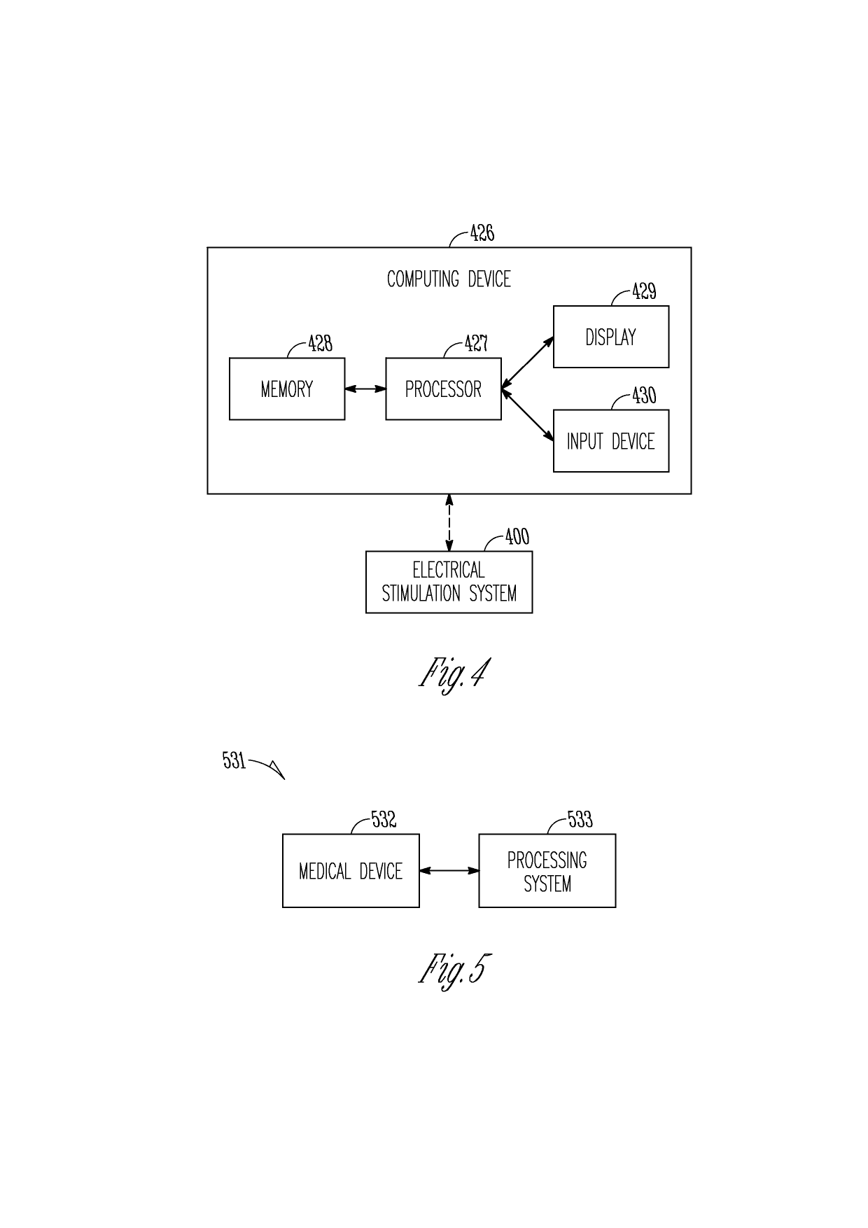

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more sets of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient’s skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracting the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson’s disease for example, the IPG 202 is typically implanted under the patient’s clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 can also be implanted underneath the scalp closer to the location of the electrodes’ implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some architectures, electrodes of the same polarity can deliver distinct amounts of current simultaneously using multiple electrical sources, to provide greater control of the electric field. In some instances, an electrode might be an anode for a period of time and a cathode for a period of time (e.g., when multiple phases are used, for example, for charge recovery or other purposes). These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering using multiple electrical sources can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length of the lead

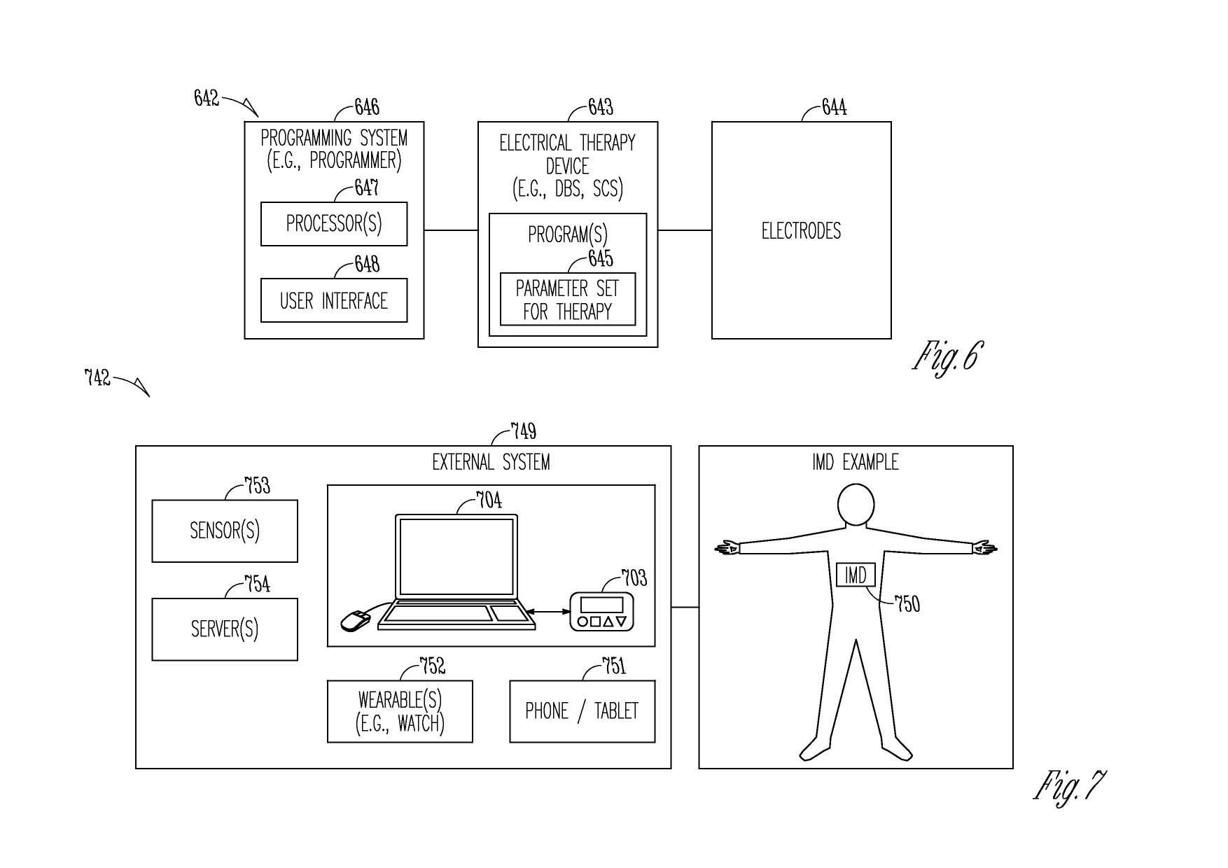

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1. Alternatively, the computing device 426 may be integrated with the electrical stimulation system 100, such as part of the IPG 102, RC 103, CP 104, or ETM 105 illustrated in FIG. 1.

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer’s disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, which is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses. In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein. The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

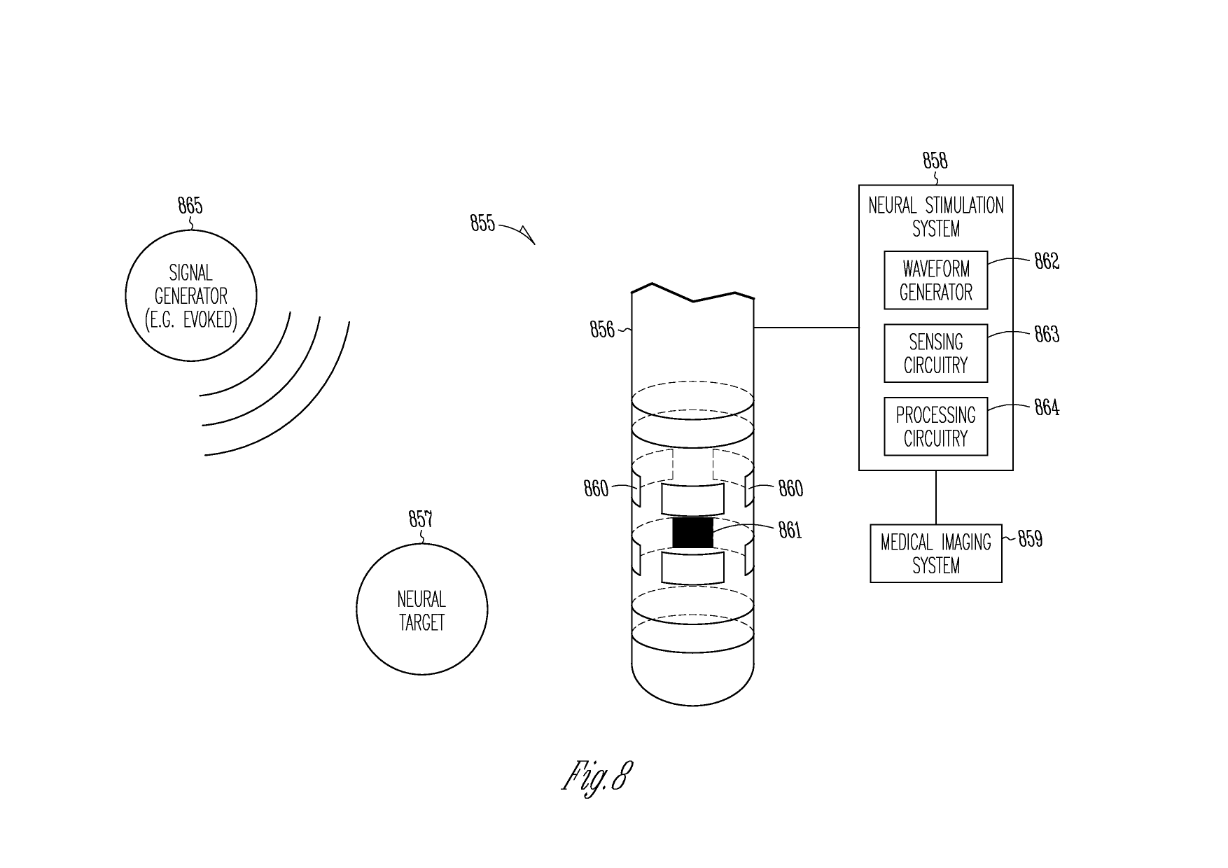

FIG. 5 illustrates, by way of example and not limitation, a more generalized example of a medical system 531 that includes a medical device 532 and a processing system 533. For example, the electrical stimulation system 400 of FIG. 4 may be a more specific example of the medical device 532 of FIG. 5, and computing device 426 of FIG. 4 may be a more specific example of the processing system 533 of FIG. 5. The medical device may be configured to use at least one directional lead to provide sensing functions and/or therapy functions. For example, the medical device may include a device configured to use a parameter set to deliver an electrical stimulation therapy. The medical device may be an implantable medical device such as an implantable neurostimulator. The implantable medical device may be configured to deliver SCS or DBS therapy. The medical device may include more than one medical device. The processing system may be within a single device or may be a distributed system across two or more devices including local and/or remote systems. According to various embodiments, the medical system may include at least one medical device configured to treat a condition by delivering a therapy to a patient.

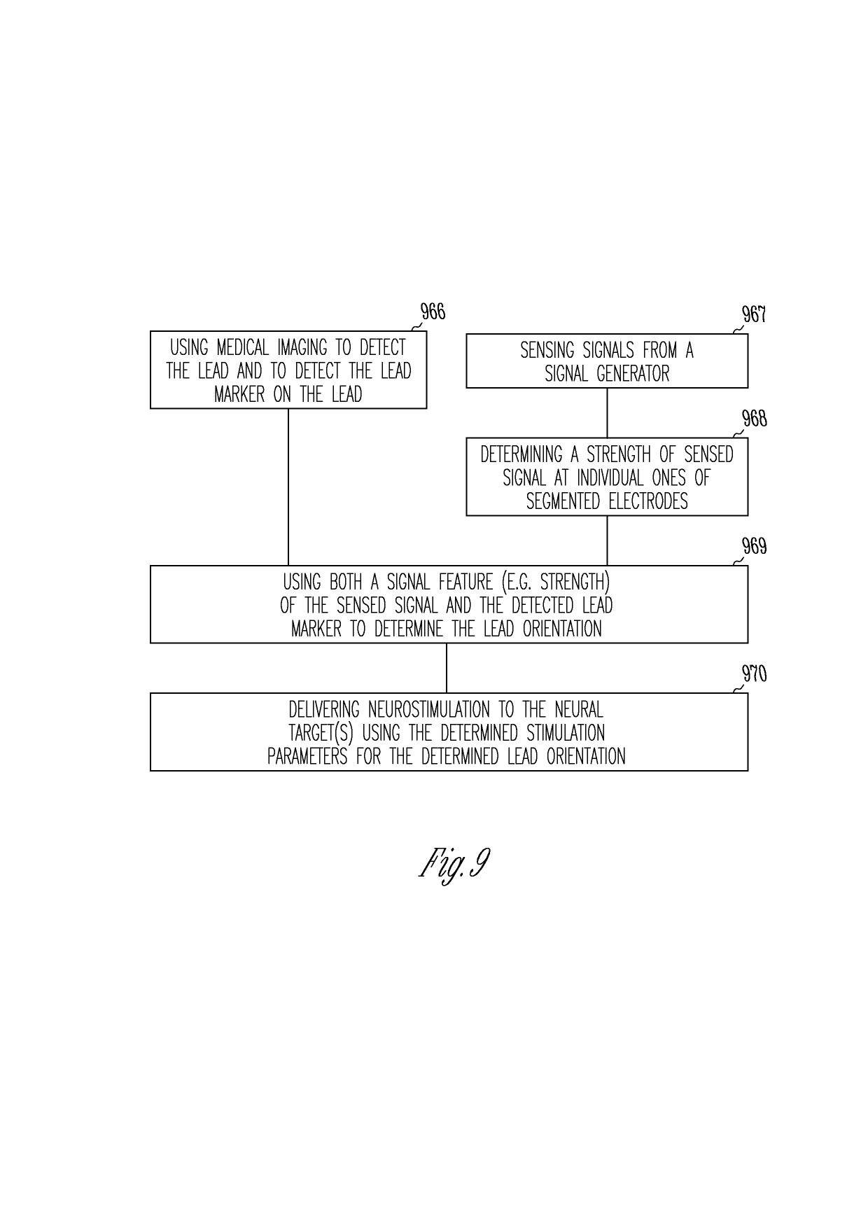

FIG. 6 illustrates, by way of example, an example of an electrical therapy-delivery system. The illustrated system 642 may be a more specific example of the system illustrated in FIG. 5 or form a portion of the system illustrated in FIG. 5. The illustrated system 642 includes an electrical therapy device 643 configured to deliver an electrical therapy to electrodes 644 to treat a condition in accordance with a programmed parameter set 645 for the therapy. The system 642 may include a programming system 646, which may function as at least a portion of a processing system, which may include one or more processors 647 and a user interface 648. The programming system 646 may be used to program and/or evaluate the parameter set(s) used to deliver the therapy. The illustrated system 642 may be a DBS system. In some embodiments, the illustrated system 642 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 7 illustrates, by way of example and not limitation, the electrical therapy-delivery system of FIG. 6 implemented using an IMD. The IMD may include a DBS stimulator. The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a remote-control device 703, similar to RC 103 in FIG. 1, configured for use by the patient to communicate with and program the neuromodulator. For example, the remote-control device 703 may allow the patient to turn a therapy ON and OFF, change or select programs, adjust patient-programmable parameter(s) of the plurality of modulation parameters, and/or provide inputs used to detect event(s). The patient may use custom external devices to perform specific tasks useful for the event-triggered therapy of that patient. The external devices may communicate directly with the IPG/IMD, or they may communicate with an intermediary device (such as an RC) which in turn communicates with the IPG/IMD. FIG. 7 illustrates an IMD 750, although the monitor and/or therapy device may be an external device such as a wearable device. The external system 749 may include a network of computers, including computer(s) remotely located from the IMD 750 that are capable of communicating via one or more communication networks with the programmer 704 and/or the remote-control device 703. The remotely located computer(s) and the IMD 750 may be configured to communicate with each other via another external device such as the programmer 704 or the remote-control device 703. The remote-control device 703 and/or the programmer 704 may allow a user (e.g., patient, caregiver and/or clinician or rep) to answer questions as part of a data collection process. The external system 749 may include personal devices such as a phone or tablet 751, wearables such as a watch 752, sensor(s) 753 and server(s)s 754. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. The system 742 may include medical record(s) for the patient and broader patient population(s). The medical record(s) may be stored and accessed using one or more servers (e.g., local or remote servers such as cloud-based servers). The external device may also include device(s) (e.g., app on phone / tablet or a custom device) used by the patient to perform tasks and may also monitor the ability of the patient to perform the task. The external system may be used to process inputs, detect events, analyze the results and/or optimize the training. Processing may be done using cloud computing, fog computing, and/or edge computing. Cloud computing may include a network of devices or servers connected over the Internet. Cloud computing may have very large storage space and processing capabilities. However, cloud computing can have higher latencies. Fog computing occurs physically closer to the end user compared to centralized data centers. The infrastructure of fog computing may connect end devices with central servers in the cloud. Fog computing may provide lower latency for quicker responses and may use other communication technology other than the Internet. Edge computing is done at the device level. The processing for different functions may be distributed over multiple devices and may be distributed over edge computing, fog computing and cloud computing.

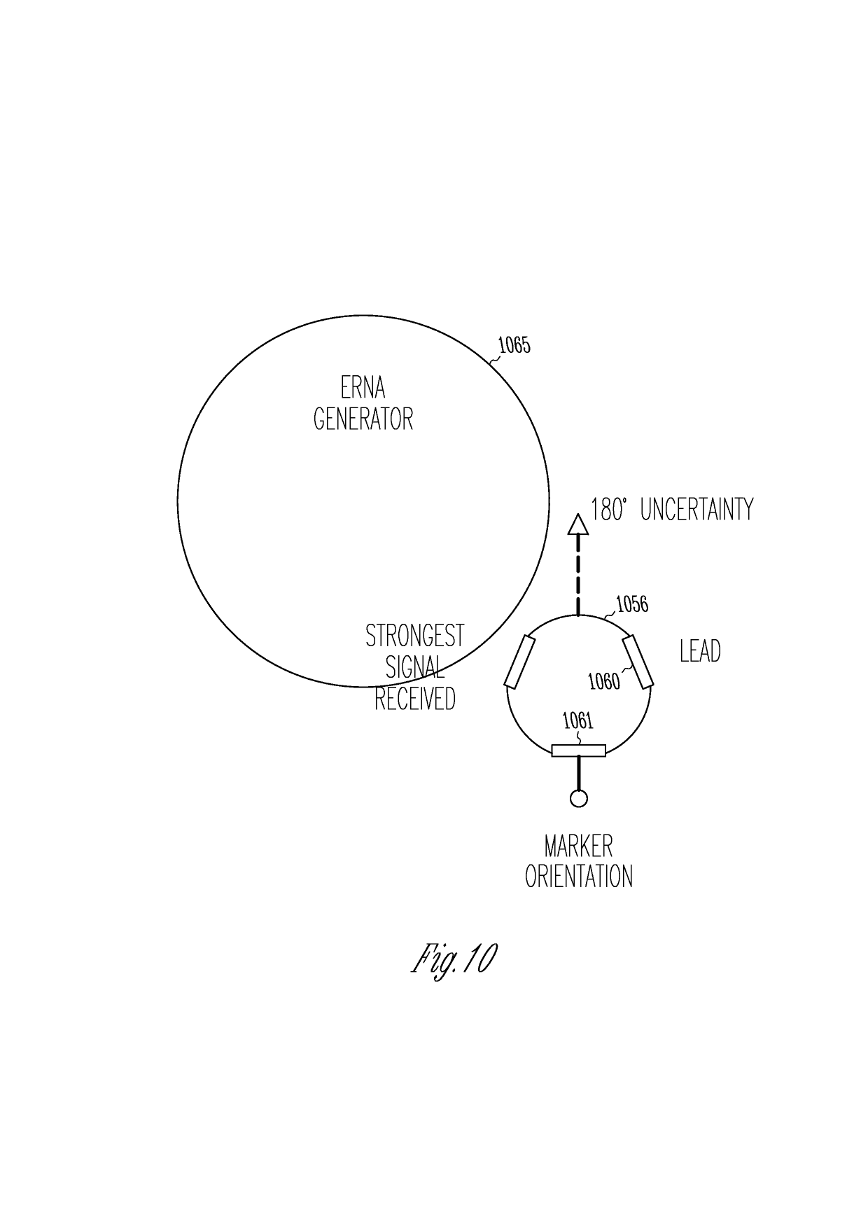

FIG. 8 illustrates, by way of example and not limitation, an embodiment of a system for determining an orientation of a neurostimulation lead for use to stimulate a neural target based on sensed signal from a signal generator. The signal generator may be a known or presumed signal generator. The signal generator may be internal or external to the patient. Internal signal generators may include machines or devices (e.g., artificial or non-physiological) or innate portions of a body that generate the signals, including various endogenous or natural signals or signal generators. Machines or devices may include components such as passive components connected to an outside world or to other machines, such as a stent connected to a signal generator. Various embodiments provided herein use sensed physiological signals from a physiological or artificial signal generator to aid in lead orientation determination. The signal generator source may be an evoked signal generator. For example, stimulation of a location in the patient may cause an evoked signal from the signal generator. The location of the signal generator may be different from the stimulated location. The evoked signal may be stimulated or sensed at various locations in the body. For example, stimulating an area of the body may evoke a neural response, may generate sensory evoked potentials, or may generate motor evoked potentials. Some embodiments may incorporate stimulation-induced side effect (e.g., facial muscle pulling below a visual perception level, as sensed using electromyography EMG). The signal generator may include a stent-mounted electrode array or another lead, for example. An internal signal may inject from the stent-mounted electrode array or from the second lead, for example.

The system 855 may include a lead 856 for stimulating neural target(s) 857 in a patient, a neural stimulator system 858 and a medical imaging system 859. Examples of medical imaging systems include fluoroscopy, computer tomography (CT), magnetic resonance imaging (MRI) and ultrasound. The lead 856 may be referred to as a directional lead and may have segmented electrodes 860 positioned peripherally around the lead and a lead marker 861 at a known position on the lead and useful for determining the orientation of the lead. The lead marker 861 may have a variety of shapes and may be made of a material that is clearly detected by the imaging technology such as a radiopaque material for x-rays. The neural stimulator system 858 may include a waveform generator 862, sensing circuitry 863, and processing circuitry 864. The neural stimulator system 858 may include one or more devices and may include components of the system illustrated in FIGS. 1, 2, 4, 6 or 7. For example, the external trial modulator 105 and clinical programmer may together provide the waveform generator 862, the sensing circuitry 863 and the processing circuitry 864.

The waveform generator 862 may be configured to deliver an electrical waveform to stimulate the at least one neural target according to neural stimulation parameters. For example, the generated waveform may be a temporal series of electrical pulses. The pulses may be delivered with a pattern. The pattern may be a simple pattern with a constant parameters such as a constant pulse amplitude, constant pulse width and constant pulse frequency, or the pattern may be more complex with different parameters. Also, the pulses may include rectilinear shapes or other shapes.

The sensing circuitry 863 may be configured to use the segmented electrodes 860 positioned peripherally around the lead to sense signals from a known signal generator 865. The known signal generator 865 has a known location distinct from a location of the at least one neural target 857 in the patient. The signal generator 865 may be an innate, natural generator in a patient that naturally provides detectible signal from a known location. Examples may include cardiovascular signals (e.g., cardiac contractions). Natural generators may include evoked signal generators where stimulation of a neural target may evoke the signals may be evoked from the generators. The evoked signals may be evoked neural responses, evoked motor potentials or sensory-evoked signals. For example, an evoked signal generator may include a generator of evoked resonant neural activity (ERNA) signals within a brain. ERNA may also be called a DLEP (DBS Local Evoked Potential), or other neural response signal spontaneously present or present as a result of the stimulation. Sensory-evoked signals may be, but is not necessarily, an evoked potential. For example, a light may be shined in a patient’s eye and a radially differentially distributed change in a non-stimulation (DBS) evoked local field potential may be detected.

The processing circuitry 864 may be configured to determine a strength of a sensed signal received at individual ones of the segmented electrodes positioned peripherally around the lead. The medical imaging system may be configured to detect the lead and to detect the lead marker on the lead. The processing circuitry may be configured to use both a signal feature (e.g., the strength or amplitude, a temporal feature and/or a spectral feature) of the sensed signal and the detected lead marker to determine the lead orientation and to determine the stimulation parameters for stimulating at least one neural target using the determined lead orientation. The system may be configured to detect simulation induced side effects, such as sensing using electromyography to detect facial muscle pulling below a visual perception level. Thus, the stimulation parameters may further be determined to avoid such side effects.

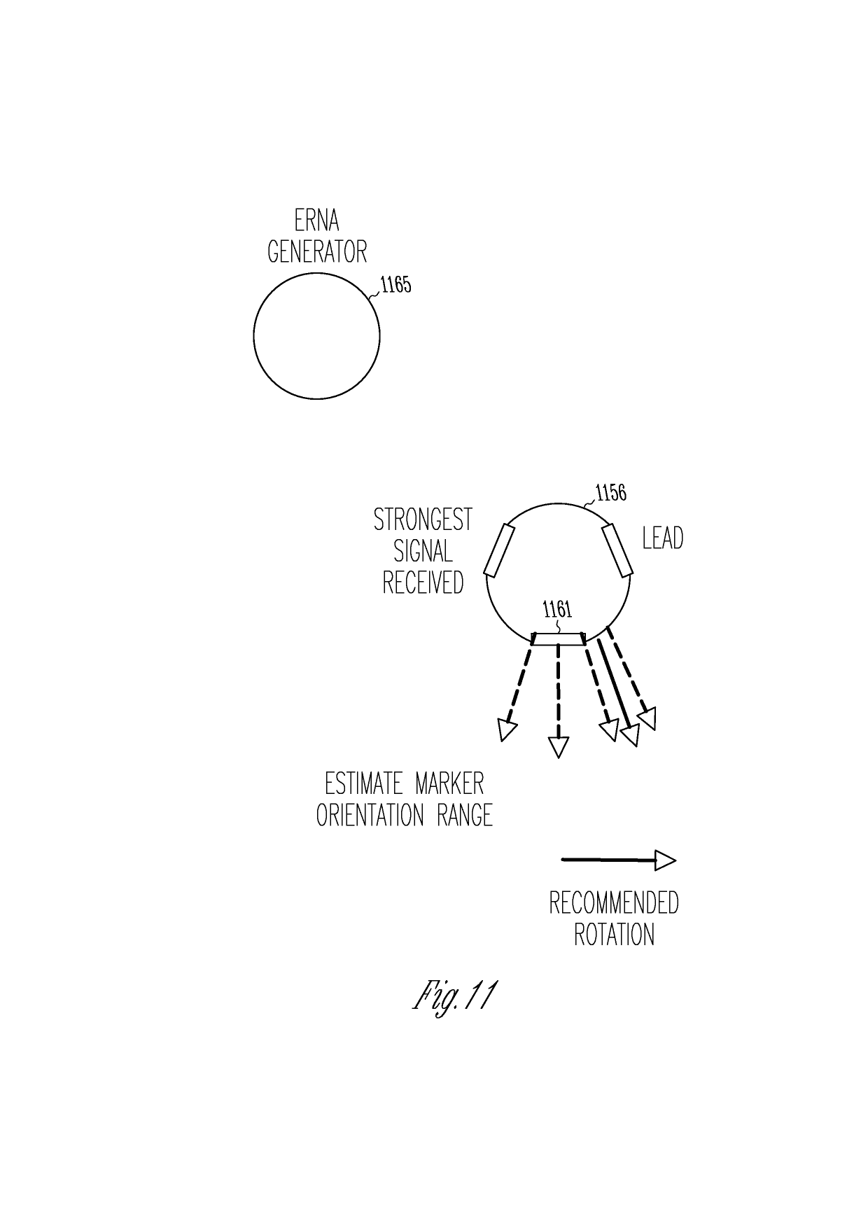

FIG. 9 illustrates, by way of example and not limitation, an embodiment of a method for determining an orientation of a neurostimulation lead for use to stimulate a neural target based on sensed signal from a signal generator. The method matter may include determining an orientation of a lead for stimulating at least one neural target in a patient and determining stimulation parameters for stimulating at least one neural target using the determined lead orientation. In a more particular example, the method may include using medical imaging to detect the lead and to detect the lead marker on the lead 966, and may also include sensing signals (e.g., ERNA) from a signal generator at a known location 967 and determining a strength of the sensed signal at individual ones of segmented electrodes 968. The lead orientation may be determined using both a feature (e.g., strength) of the sensed signal and the detected lead marker 969. For example, a segmented electrode closest toward the signal generator has a largest sensed signal. Some embodiments may simply use the largest sensed signal or use all the sensed signals from the segmented electrodes and determined the lead orientation by comparing to templates or using a model for the sensed signals and the implanted lead. Alternatively, or additionally, other signal features such as temporal and/or spectral features) may be used. At 970, neurostimulation may be delivered to the neural target(s) using the determined stimulation parameters for the determined lead orientation. For example, the appropriate ones of the electrodes on the lead may be activated, and the anodic and cathodic energy may be fractionalized across the active electrodes to provide the desired stimulation field toward the neural target(s).

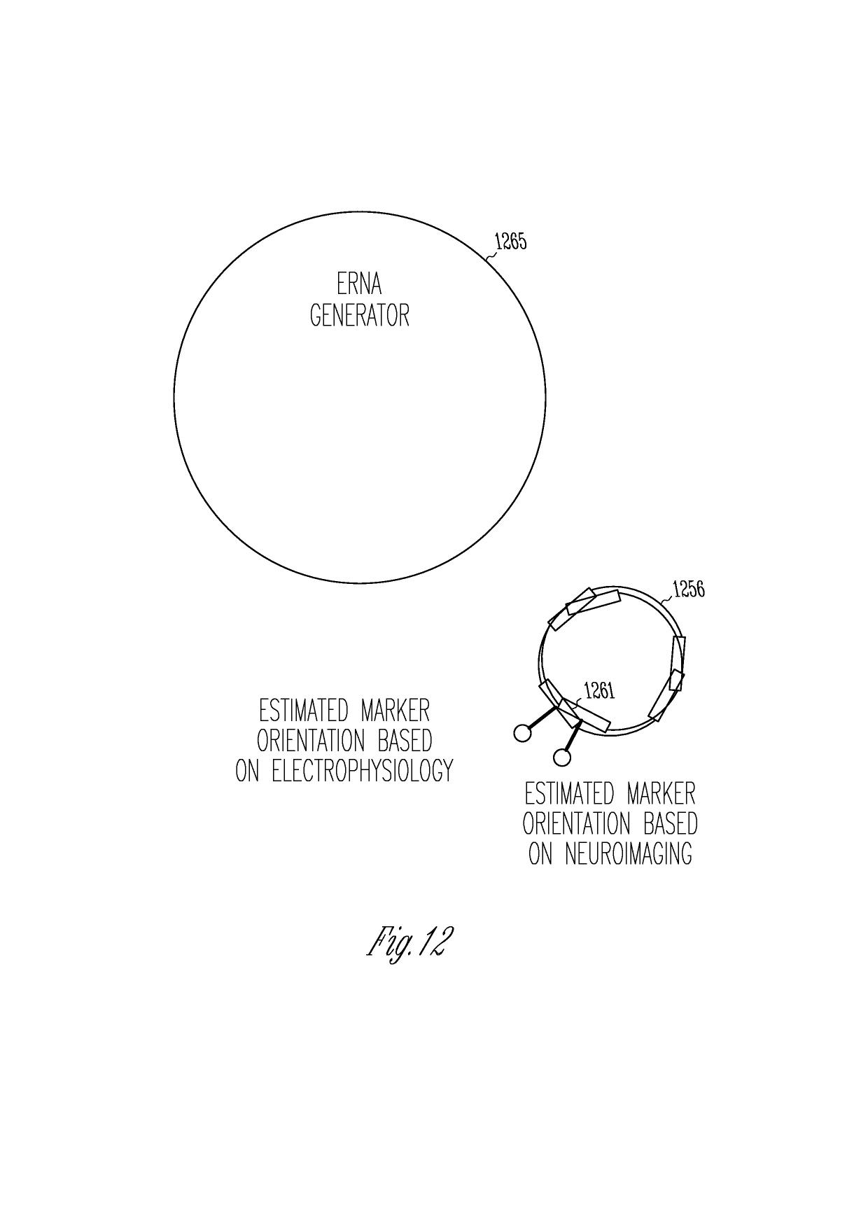

FIG. 10 illustrates, by way of example and not limitation, an uncertainty in the marker position based solely on medical imaging, and the use of a signal generator such as an evoked resonant neural activity (ERNA) generator to resolve the uncertainty. The lead 1056 includes segmented electrodes 1060 and further includes a lead marker 1061. However, medical imaging alone may not be able to correctly detect the orientation of the lead marker 1061 on the lead 1056. In the illustrated example, the algorithms that interpret medical imaging may make a 180° error as it may not know if the lead marker is on a near surface (e.g., toward bottom of figure) or far surface (e.g., toward top of figure) of the lead 1056. This uncertainty may be reduced or eliminated by sensing a signal from a fixed source 1065. The neural response signal may be an EP signal, an ERNA signal also called a DLEP (DBS Local Evoked Potential), or other neural response signal spontaneously present or present as a result of the stimulation. The fixed source may be an anatomical source as some sensed signals originate from relatively consistent anatomical sources. A specific example for DBS lead placement is an ERNA generator. The sensed ERNA signals are expected to be stronger when recorded from one angle of the lead, such that fixed anatomical signal can be used to help orient the lead. In the illustrated embodiment, the ERNA generator may be known to be in a Posterior Medial location relative to the target structure where the lead is implanted. The user may use current algorithms to identify where the ERNA signal is strongest and if the lead is in a good position. Secondarily, an additional algorithm may reduce or eliminate the uncertainty by verifying that, based on the recorded signal, the lead marker must be oriented anteriorly. The 180° uncertainty is a specific example. The difference in lead rotation from the intended anterior orientation is often between 40 to 50 degrees. Since the intended orientation is anterior. the orientation estimation algorithm may automatically consider the estimation closest to the desired orientation and discard the 180° degree alternative.

FIG. 11 illustrates, by way of example and not limitation, the use of a signal generator such as an ERNA generator to estimate a marker orientation and provide a recommendation for rotating the lead. In some instances, it may be useful to estimate an orientation of the lead marker 1161 prior to obtaining imaging data. This may allow the neurosurgeon to rotate the lead 1156 to a preferred angle if the lead is currently pointing in another direction. The algorithm may function similar to the last where the position along the lead where the strongest ERNA signal from the ERNA generator 1165 is sensed is used to orient the lead relative to the anatomy. The estimated marker position range may be calculated, and based on this estimate, the algorithm performed the processing circuitry may recommend how to rotate the lead to most likely center around the physician's preferred orientation. This recommendation may be quickly provided as the lead is being advanced into position to stimulate the neural target(s). The algorithm performed by the processing circuitry may be configured to account for orientation in post-procedure workflows and algorithms.

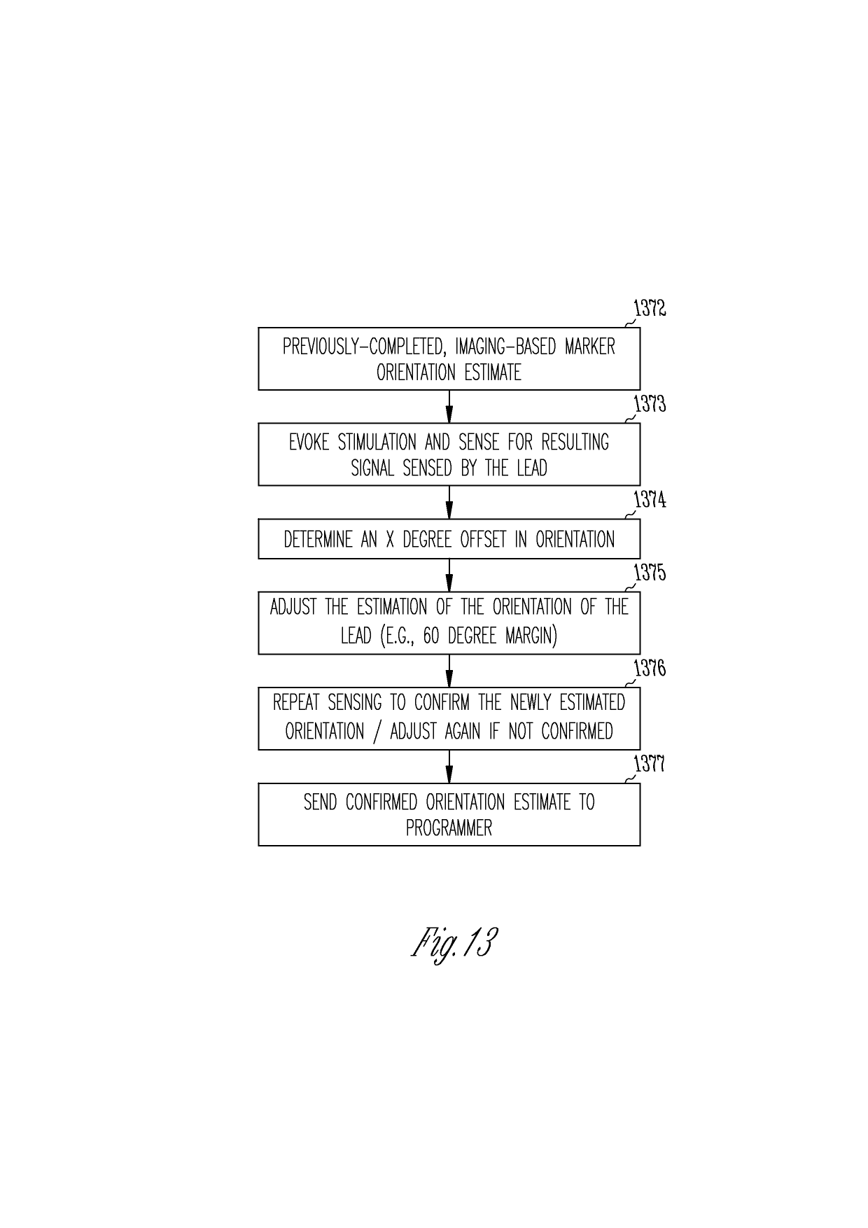

FIG. 12 illustrates, by way of example and not limitation, an estimated lead orientation based on medical imaging and a different estimated lead orientation based on sensed electrophysiological signals from a signal generator at a known location. Radiographical (neuroimaging) data is used to provide a first estimation of the orientation of the lead 1256, and the orientation of the lead marker 1261 may be used to provide a second estimation of the orientation of the lead based on the sensed electrophysiology (i.e., sensed ERNA from the ERNA generator 1265). Both estimations may be used to determine a lead orientation. By way of example and not limitation, the first estimation of the lead orientation may be adjusted using the second estimation, or the second estimation may be adjusted using the first estimation.

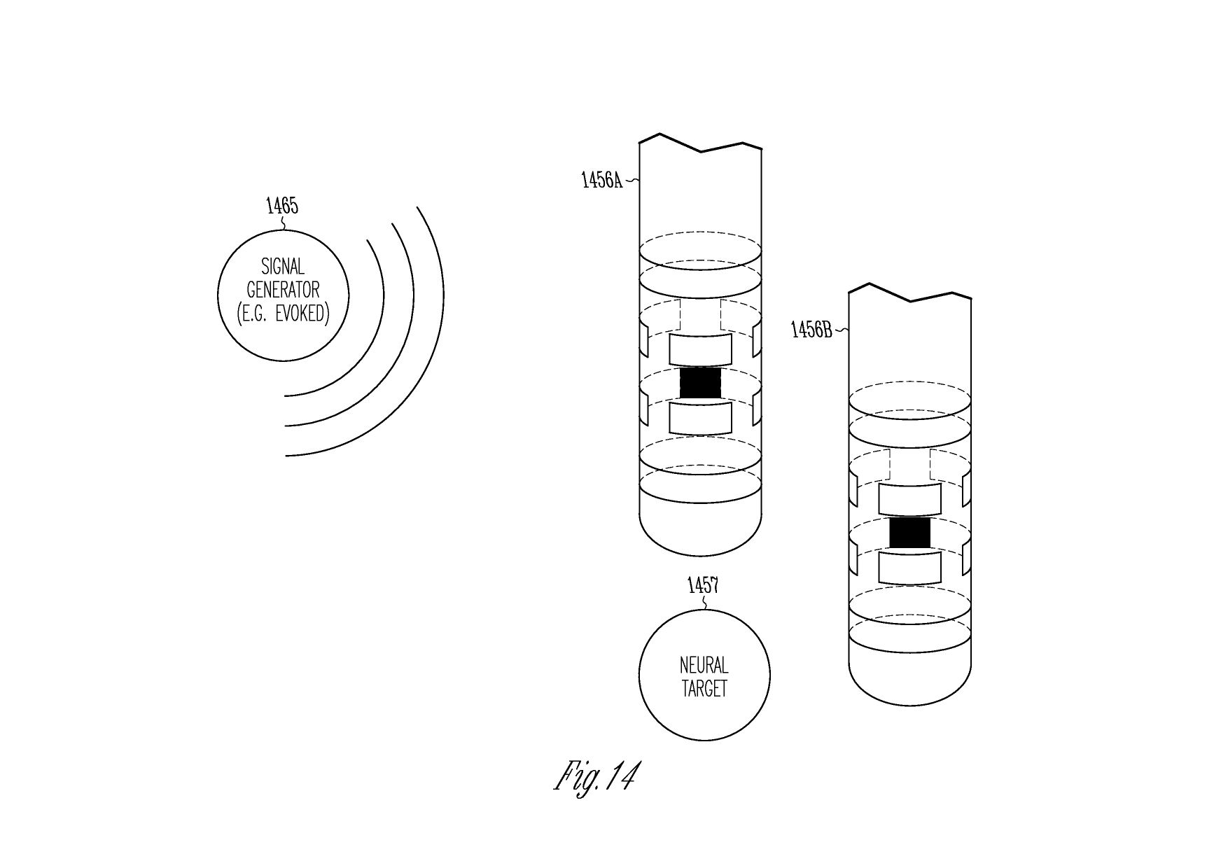

FIG. 13 illustrates, by way of example and not limitation, an embodiment of a method for revising an image-determined orientation estimate using a sensed signal sensed by the lead. A previously-completed imaging-based marker orientation estimate may be accessed at 1372. At 1373, stimulation and sensing may be performed to sense the evoked signal at the lead. An offset (e.g., X degrees) in orientation may be determined 1374. The estimation of the orientation of the lead may be adjusted (e.g., 60 degree margin) 1375. The sensing may be repeated to confirm the newly estimated orientation, and further refinements or adjustments may be performed if the newly estimated orientation was not able to be confirmed 1376. By way of example and not limitation, a first suggested orientation may be between 30 and 60 degrees, and this first suggested orientation may be refined to between 45 and 50 degrees. The confirmed orientation estimate may be sent to the programmer for use to determine the stimulation parameters for the directional lead to stimulate the neural target(s) 1377.

FIG. 14 illustrates, by way of example and not limitation, an embodiment of a system for determining an orientation of a neurostimulation lead for use to stimulate a neural target based on sensed signal from a signal generator as sensed by one or both of a first lead or a second lead. The relative position between the two or more leads 1456A and 1456B may be determined (e.g., U.S. Pat. Nos. 7,684,869 and 7,853,330, which are incorporated by reference in their entirety). Each one of these leads can also detect the signals from the signal generator (e.g., ERNA generator 1465), which may be used along with knowledge of the relative position of the leads to detect the orientation of the leads more accurately. Typically, leads are implanted bilaterally. Information from contralateral leads can be used to inform and refine orientation. Occasionally, more than one lead is implanted per hemisphere. Information from same-hemisphere, different-(surgical) target leads can be used to inform, refine orientation.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
determining an orientation of a lead for stimulating at least one neural target in a patient, wherein the lead includes segmented electrodes positioned peripherally around the lead and a lead marker at a known position on the lead and useful for determining the orientation of the lead, and wherein the determining the orientation of the lead includes:
using medical imaging to detect the lead and to detect the lead marker on the lead;
sensing signals from a signal generator using the segmented electrodes positioned peripherally around the lead, wherein the signal generator has a known location distinct from a location of the at least one neural target in the patient;
determining a strength of a sensed signal received at individual ones of the segmented electrodes positioned peripherally around the lead; and
using both the strength of the sensed signal and the detected lead marker to determine the lead orientation; and
determining stimulation parameters for stimulating the at least one neural target using the determined lead orientation.

2. The method of claim 1, wherein the signal generator is a known evoked response generator in the patient and the signals sensed from the signal generator include evoked response signals, and the method further includes causing the known evoked response generator to generate the evoked response signals.

3. The method of claim 2, wherein the lead is used to deliver stimulation to cause the known evoked response generator to generate the evoked response signals.

4. The method of claim 3, wherein the generated evoked responses include evoked motor potentials.

5. The method of claim 3, wherein the generated evoked responses include evoked neural responses.

6. The method of claim 3, wherein the generated evoked responses include sensory evoked potentials.

7. The method of claim 1, wherein the lead includes a deep brain stimulation (DBS) lead configured for implantation in a brain, and the location of the signal generator is in the brain.

8. The method of claim 1, wherein the lead includes a deep brain stimulation (DBS) lead configured for implantation in a brain and the signals include evoked resonant neural activity (ERNA) in the brain.

9. The method of claim 1, further comprising delivering neurostimulation to the at least neural target using the determined stimulation parameters for the determined lead orientation.

10. The method of claim 1, wherein the lead marker is detected using medical imaging before the signals are sensed.

11. The method of claim 10, wherein the detected lead marker on the lead is used to provide a suggested orientation of the lead, and the sensed signals from the known position of the signal generator are used to confirm the suggested orientation of the lead or modify the suggested orientation of the lead.

12. The method of claim 1, wherein the signals are sensed before the lead marker is detected using medical imaging.

13. The method of claim 12, further comprising automatically providing a recommendation for rotating the lead during a lead implantation procedure.

14. The method of claim 1, further comprising using the medical imaging to implant the lead into position to stimulate the at least one neural target.

15. The method of claim 14, further comprising orientating the lead into a desired orientation using both the strength of the sensed signal and the detected lead marker while the lead is being implanted.

16. The method of claim 1, wherein the lead is a first lead, the method further comprising sensing signals from the signal generator using segmented electrodes positioned peripherally around a second lead, wherein the lead orientation for the first lead is determined using the signals sensed using the second lead.

17. The method of claim 16, wherein the first lead is a first DBS lead and the second lead is a second DBS lead, and the first DBS lead and the second DBS lead are contralateral leads.

18. The method of claim 16, wherein the first lead is a first DBS lead and the second lead is a second DBS lead, and the first DBS lead and the second DBS lead are in a same brain hemisphere.

19. A non-transitory machine-readable medium including instructions, which when executed by a machine, cause the machine to perform a method comprising:
determining an orientation of a lead for stimulating at least one neural target in a patient, wherein the lead includes segmented electrodes positioned peripherally around the lead and a lead marker at a known position on the lead and useful for determining the orientation of the lead, and wherein the determining the orientation of the lead includes:
receiving lead marker information indicative of a detected lead marker on the lead that was detected using medical imaging;
sensing signals from a signal generator using the segmented electrodes positioned peripherally around the lead, wherein the signal generator has a known location distinct from a location of the at least one neural target in the patient;
determining a strength of a sensed signal received at individual ones of the segmented electrodes positioned peripherally around the lead; and
using both the strength of the sensed signal and the detected lead marker to determine the lead orientation; and
determining stimulation parameters for stimulating the at least one neural target using the determined lead orientation.

20. A system, comprising:
a lead including segmented electrodes positioned peripherally around the lead;
an orientation system configured to determine an orientation of the lead implanted in a patient, the orientation system including:
sensing circuitry configured to use the segmented electrodes positioned peripherally around the lead to sense signals from a signal generator; and
processing circuitry configured to determine at least one feature of a sensed signal received at individual ones of the segmented electrodes positioned peripherally around the lead and to use the at least one feature at the individual ones of the segmented electrodes to determine lead orientation.
